(19) United States
(12) Patent Application Publication
(10) Pub. No.: US 2026/0083441 A1
(43) Pub. Date: Mar. 26, 2026

(54) BIOLOGICAL SAMPLE COLLECTION SYSTEM WITH TWIST-ACTIVATED VALVE FOR SAMPLE RELEASE

(71) Applicant: DNA GENOTEK INC., Kanata, Ontario (CA)

(72) Inventor: Attila Csaba NEMETH, White Lake (CA)

(73) Assignee: DNA GENOTEK INC., Kanata, Ontario (CA)

(21) Appl. No.: 19/106,737

(22) PCT Filed: Aug. 25, 2023
(86) PCT No.: PCT/CA2023/051127

§ 371 Date: Feb. 26, 2025

Related U.S. Application Data
(60) Provisional application No. 63/401,463, filed on Aug. 26, 2022.

(51) Int. Cl. A61B 10/00

(52) U.S. Cl. CPC A61B 10/0096

(57) ABSTRACT

A biological sample collection system comprising a containment vessel and a sample collection assembly connected thereto is provided. The assembly comprises a sample collector threadedly engaged with a sample collection vessel having a sample receiving area for receiving the biological sample from the sample collector. The collector comprises a valve stem comprising a valve face, and the collection vessel comprises a complementary valve seat. The valve stem and valve seat form a valve comprising a closed configuration wherein threaded connection members of the collector and the collection vessel are fully engaged, and an open configuration wherein the threaded connection members are less than fully engaged. In the closed configuration, the valve face forms a sealing engagement with the valve seat. In the open configuration, the valve face is spaced apart from the valve seat to place the containment vessel, collection vessel, and collector in fluid communication with one another.

### FIELD OF THE INVENTION

The present invention pertains to a biological sample collection system. More particularly, the present invention pertains to a biological sample collection system with a twist-activated valve for sample release.

### BACKGROUND

Field collections of biological samples, such as saliva or sputum, can provide invaluable information about the patient or donor. It can be inconvenient to require patients/donors to travel to a biological sample collection site or laboratory. Similarly, it may be difficult and costly for laboratory personnel to directly access the patient/donor for sample collection, particularly if the sample size is large and/or geographically diverse. In addition, patients/donors are often inexperienced, first-time donors, so it is critical that the biological sample collection system be easy to operate or manipulate by novice users. A desirable collection system removes the need for specialized personnel/clinicians, facilities and equipment when collecting, transporting and storing biological samples.

At ambient temperature, nucleic acids and other biomolecules (e.g. metabolites) in biological samples quickly degrade and must generally be stored under freezing temperatures to remain stable. This problem is amplified when a biological sample is collected at a remote field site, or a significant distance from the laboratory or doctor's office, and especially where power and freezers are non-existent or not constant. In addition, shipping biological samples on dry ice or ice packs is costly, complicated, and impractical and could lead to the exposure of workers to infectious agents or pathogens. Hence, there is a need for a safe collection, transport and storage system that maintains the integrity of the collected biomolecules in biological samples under ambient conditions for further molecular analysis or diagnostic testing.

There is a need for an improved container system for releasably and reliably storing a substance. It is often desirable to store a substance, such as a liquid, solid, slurry, gas, mixtures thereof, or the like, in a container or compartment prior to mixing the contents of the container with another material. For example, it may be desirable to package and store a compound, or compounds, in a container for shipping and/or safe storage and handling, prior to combining the compound(s) with another material.

It may be desirable to package and store a toxic compound in a container, prior to combining such a toxic compound with a detoxifying material or reagent. As well, it is often desirable to keep a concentrated active ingredient separate from a diluent until immediately prior to use. Container systems having a separate, sealed compartment/chamber for the sample preservative protect the patient/specimen donor from contacting, ingesting and/or spilling the preservative. Safely confining the toxic compound or preservative within a separate compartment of the sample collection device or system allows the use of more potent chemicals or preservatives (e.g. denaturants) to stabilize biomarkers/biomolecules, e.g. nucleic acids and metabolites, contained in the collected specimen. Only after the device or system containing the biological specimen is closed with a lid or cap does the compartment for the preservative open and allow mixing of the biological specimen with the preservative.

There are a variety of containers/systems for holding substances separately in such a manner that a user may seal a closure to combine the substances. Typically, these containers are double compartment systems in which substances are stored separately and substances are combined by application of the container/system closure by a user. There are numerous ways to design said two compartment/chamber systems. In particular, there is a need for a patient-friendly sample collection device or system that allows for safe at-home collection, followed by leak-proof transportation and storage under ambient or room temperature conditions. Two compartment/chamber systems are ideal for protecting the donor/patient from the preservative needed to preserve the sample or analyte of interest until analysis. Ideally, the method or instructions involve a minimal number of steps for use.

There is a need for biological sample collection systems to facilitate sample collection.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In one aspect, there is provided a biological sample collection system. The system comprises a containment vessel comprising a first open end for receiving a biological sample, a second end comprising a sample storage chamber, and a connection member disposed at the first open end; and, a sample collection assembly that is configured to be connected to the first open end of the containment vessel when the biological sample is being collected. The sample collection assembly comprises a sample collector comprising a proximal open end for receiving a biological sample, a distal open end in fluid communication with the proximal open end, a valve stem, and a threaded connection member disposed at the distal open end of the sample collector. The valve stem comprises a first stem end extending from an inner portion of the sample collector, the inner portion being disposed proximal to the distal open end of the sample collector, and an opposing second stem end comprising a valve face, the valve stem extending along a longitudinal axis of the sample collector and in a direction away from the proximal open end of the sample collector. The sample collection assembly further comprises a sample collection vessel configured to be in fluid communication with the sample collector when the biological sample is being collected, the sample collection vessel comprising a proximal open end for receiving the biological sample from the sample collector, a distal open end configured to be in fluid communication with the proximal open end, one or more walls defining a sample receiving area and a valve seat, wherein the valve seat is complementary to the valve face of the sample collector. The sample collection assembly further comprises a threaded connection member disposed at the proximal open end, wherein the threaded connection member is complementary to and configured to be engaged with the threaded connection member of the sample collector, and a connection member disposed at the distal open end, wherein the connection member is complementary to and configured to be engaged with the connection member of the containment vessel. The valve stem and the valve seat form a valve comprising a closed configuration wherein the threaded connection members of the sample collector and the sample collection vessel are fully engaged, wherein when the valve is in the closed configuration the valve face forms a sealing engagement with the valve seat; and, an open configuration wherein the threaded connection members of the sample collector and the sample collection vessel are less than fully engaged, wherein when the valve is in the open configuration the valve face is spaced apart from the valve seat such that the containment vessel, the sample collection vessel, and the sample collector are in fluid communication with one another.

In another aspect, there is provided a method of preserving a biomolecule and/or neutralizing one or more inhibitors in a biological sample, the method comprising: a) obtaining a biological sample; b) obtaining the above-noted biological sample collection system, wherein the valve is in the closed configuration; c) placing the biological sample in the sample collector, the sample collector being in fluid communication with the sample collection vessel such that the biological sample is received in the sample receiving area of the sample collection vessel; d) rotating the sample collector in a first direction (e.g. counter-clockwise) relative to the sample collection vessel to move the valve from the closed configuration to the open configuration, such that the containment vessel, the sample collection vessel, and the sample collector are in fluid communication with one another and the biological sample is received in the sample storage chamber of the containment vessel; e) optionally, rotating the sample collector in a second direction (e.g. clockwise) relative to the sample collection vessel to return the valve to the closed configuration, wherein the second direction is opposite to the first direction; and f) mixing the biological sample received in the sample storage chamber of the containment vessel with a stabilizing composition, a preservative, and/or a neutralizing agent in the containment vessel for preserving the biomolecule and/or neutralizing one or more inhibitors within the biological sample.

### BRIEF DESCRIPTION OF THE FIGURES

For a better understanding of the present invention including the progression of development to get to the end product, reference is made to the following description which is to be used in conjunction with the accompanying drawings, where:

FIG. 1 illustrates a perspective, exploded view of a biological sample collection system 100 in accordance with one embodiment of the present application.

FIG. 2a illustrates a side, exploded view of the biological sample collection system 100 of FIG. 1. FIG. 2b is a simplified cross-sectional view taken along line C-C of FIG. 2a.

FIG. 3a illustrates a side view of the assembled biological sample collection system 100 of FIG. 1. FIG. 3b shows a simplified cross-sectional view taken along line A-A of FIG. 3a.

FIG. 4a illustrates a perspective view of the sample collector 114 of the biological sample collection system 100 of FIG. 1. FIG. 4b illustrates a top view of the sample collector 114 of FIG. 4a.

FIG. 5 illustrates a side view of an alternate embodiment of a sample collector 114 that can be used in the biological sample collection system 100 of the present application.

FIGS. 6a-d illustrate various views of an alternate embodiment of a sample collector 114 that can be used in the biological sample collection system 100 of the present application. FIG. 6a illustrates a perspective view, FIG. 6b illustrates a second perspective view, FIG. 6c illustrates a side view, and FIG. 6d illustrates a top view of the alternate embodiment of the sample collector 114.

FIGS. 7a-d illustrate various views of the sample collection vessel 114 of the biological sample collection system 100 of FIG. 1. FIG. 7a is a side view, FIG. 7b is a simplified cross-sectional view taken along line B-B of FIG. 7a, FIG. 7c is a perspective view, and FIG. 7d is a top view.

FIGS. 8a and 8b illustrate simplified cross-sectional views of the biological sample collection system 100 of FIG. 1 in operation, showing the valve in a closed configuration (FIG. 8a) and in an open configuration (FIG. 8b).

FIGS. 9a, 9b and 9c illustrate partial simplified cross-sectional views of a biological sample collection system 100a having an alternate embodiment of the sample collector, designated as 114a.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

The term “biological sample” as used herein will be understood to mean any biological specimen that potentially contains a substance of interest, in particular a nucleic acid, and optionally a protein or other biomolecules of interest, or a composition comprising such a biological specimen (e.g. in instances where a biological specimen has been pre-treated with a stabilizing/preserving agent). A biological sample can include a bodily fluid (e.g. sputum or saliva) or a composition comprising a bodily fluid. In one embodiment, the biological sample is a liquid biological sample.

The term “bodily fluid” as used herein will be understood to mean a naturally occurring fluid from a human or an animal, and includes, but is not limited to urine, saliva, sputum, serum, plasma, blood, pharyngeal, nasal/nasal pharyngeal and sinus secretions, mucous, gastric juices, pancreatic juices, bone marrow aspirates, cerebral spinal fluid, feces, semen, products of lactation or menstruation, cervical secretions, vaginal fluid, tears, or lymph. In one embodiment, the bodily fluid is selected from sputum or saliva.

The term “ambient temperature” as used herein refers to a range of temperatures that could be encountered by the biological sample collection system as described herein, which can contain a mixture of a bodily fluid (e.g. saliva sample) and a stabilizing composition/preservative/neutralizing agent, from the point of collection, during transport (which can involve relatively extreme temperatures, albeit usually for shorter periods of time (e.g. <5 days)), as well as during prolonged storage prior to analysis. In one embodiment, the ambient temperature is ranging from about −20° C. to about 50° C. In another embodiment, the ambient temperature is room temperature (RT) and ranges from about 15° C. to about 25° C.

As noted above, two compartment/chamber systems are ideal for protecting the donor/patient from the preservative needed to preserve the sample or analyte of interest until analysis. One mechanism to separate two compartments within a collection system is the utilization of a valve.

As outlined in further detail below, the system as described herein is a two compartment/chamber system that makes use of a valve to separate a sample collection vessel from a sample storage chamber, wherein the valve is moved to an open position following collection of a biological sample.

In one embodiment, there is provided a biological sample collection system. The system comprises a containment vessel comprising a first open end for receiving a biological sample, a second end comprising a sample storage chamber, and a connection member disposed at the first open end; and, a sample collection assembly that is configured to be connected to the first open end of the containment vessel when the biological sample is being collected. The sample collection assembly comprises a sample collector comprising a proximal open end for receiving a biological sample, a distal open end in fluid communication with the proximal open end, a valve stem, and a threaded connection member disposed at the distal open end of the sample collector. The valve stem comprises a first stem end extending from an inner portion of the sample collector, the inner portion being disposed proximal to the distal open end of the sample collector, and an opposing second stem end comprising a valve face, the valve stem extending along a longitudinal axis of the sample collector and in a direction away from the proximal open end of the sample collector. The sample collection assembly further comprises a sample collection vessel configured to be in fluid communication with the sample collector when the biological sample is being collected, the sample collection vessel comprising a proximal open end for receiving the biological sample from the sample collector, a distal open end configured to be in fluid communication with the proximal open end, one or more walls defining a sample receiving area and a valve seat, wherein the valve seat is complementary to the valve face of the sample collector. The sample collection assembly further comprises a threaded connection member disposed at the proximal open end, wherein the threaded connection member is complementary to and configured to be engaged with the threaded connection member of the sample collector, and a connection member disposed at the distal open end, wherein the connection member is complementary to and configured to be engaged with the connection member of the containment vessel. The valve stem and the valve seat form a valve comprising a closed configuration wherein the threaded connection members of the sample collector and the sample collection vessel are fully engaged, wherein when the valve is in the closed configuration the valve face forms a sealing engagement with the valve seat; and, an open configuration wherein the threaded connection members of the sample collector and the sample collection vessel are less than fully engaged, wherein when the valve is in the open configuration the valve face is spaced apart from the valve seat such that the containment vessel, the sample collection vessel, and the sample collector are in fluid communication with one another. In one embodiment, the valve seat is disposed towards the distal open end of the sample collection vessel.

In another embodiment, the valve stem extends centrally from the distal open end of the sample collector, the inner portion of the sample collector being centrally disposed within the sample collector, the inner portion of the sample collector being connected to an inner surface of the sample collector by at least one attachment; the inner surface of the sample collector, the inner portion of the sample collector, and the at least one attachment collectively defining at least one opening to provide fluid communication between the distal open end of the sample collector and the proximal open end of the sample collection vessel when the biological sample is being collected.

In another embodiment, the sealing engagement between the valve face and the valve seat is fluid-tight.

In yet another embodiment, the sample collector is funnel-shaped.

In still yet another embodiment, at least a portion of the sample collector extending from the proximal open end comprises a flexible material.

In another embodiment, the flexible material comprises silicone or rubber.

In yet another embodiment, the connection member disposed at the first open end of the containment vessel and the connection member disposed at the distal open end of the sample collection vessel comprise a threaded connection.

In another embodiment, the opposing second stem end extends beyond the distal open end of the sample collector.

In another embodiment, the sample receiving area of the sample collection vessel has a capacity of from about 0.1 mL to about 10 mL of sample.

In yet another embodiment, the sample storage chamber of the containment vessel comprises a stabilization composition, preservative, and/or neutralizing agent. Suitable stabilizing compositions, preservatives, and neutralizing agents are well known to the skilled worker. In another embodiment, the preservative can be a single component or a composition. In another embodiment, the neutralizing agent can be a single component or can be a composition. In another embodiment, the neutralizing agent neutralizes one or more inhibitors in a biological sample, such as nucleases, including deoxyribonucleases and ribonucleases. In another embodiment, the stabilizing composition, preservative and/or neutralizing agent can include compositions such as those described in, for example, U.S. Pat. Nos. 7,482,116, 10,000,795, or 8,158,357.

In still yet another embodiment, the valve is configured to be moved from the closed configuration to the open configuration by rotating the sample collector in a first direction (e.g. counter-clockwise) relative to the sample collection vessel, such that the threaded connection members of the sample collector and the sample collection vessel are less than fully engaged. In another embodiment, rotating the sample collector in the first direction relative to the sample collection vessel comprises a rotation of about one-quarter turn.

In another embodiment, the valve can be returned to the closed configuration from the open configuration by rotating the sample collector in a second direction (e.g. clockwise) relative to the sample collection vessel to fully engage the threaded connection members of the sample collector and the sample collection vessel, wherein the second direction is opposite to the first direction. In yet another embodiment, rotating the sample collector in the second direction relative to the sample collection vessel comprises a rotation of about one-quarter turn.

In another embodiment, there is provided a method of preserving a biomolecule and/or neutralizing one or more inhibitors in a biological sample, the method comprising: a) obtaining a biological sample; b) obtaining a biological sample collection system as described herein, wherein the valve is in the closed configuration; c) placing the biological sample in the sample collector, the sample collector being in fluid communication with the sample collection vessel such that the biological sample is received in the sample receiving area of the sample collection vessel; d) rotating the sample collector in a first direction (e.g. counter-clockwise) relative to the sample collection vessel to move the valve from the closed configuration to the open configuration, such that the containment vessel, the sample collection vessel, and the sample collector are in fluid communication with one another and the biological sample is received in the sample storage chamber of the containment vessel; e) optionally, rotating the sample collector in a second direction (e.g. clockwise) relative to the sample collection vessel to return the valve to the closed configuration, wherein the second direction is opposite to the first direction; and f) mixing the biological sample received in the sample storage chamber of the containment vessel with a stabilizing composition, a preservative, and/or a neutralizing agent in the containment vessel for preserving the biomolecule and/or neutralizing one or more inhibitors within the biological sample.

In another embodiment, rotating the sample collector in the first direction relative to the sample collection vessel comprises a rotation of about one-quarter turn. In another embodiment, rotating the sample collector in the second direction relative to the sample collection vessel comprises a rotation of about one-quarter turn.

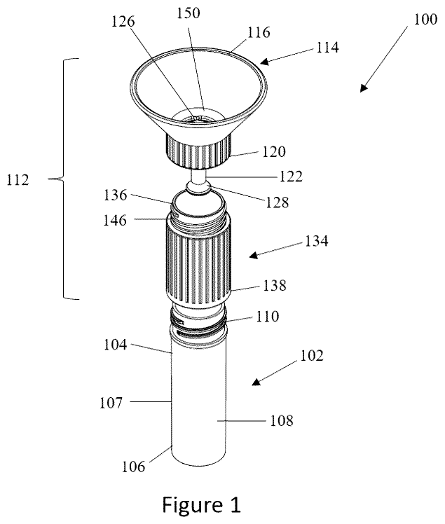

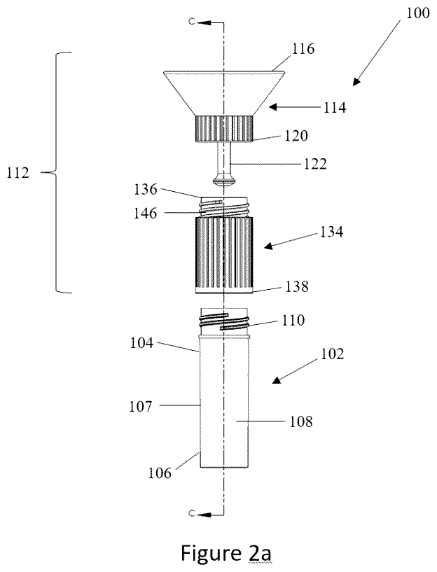

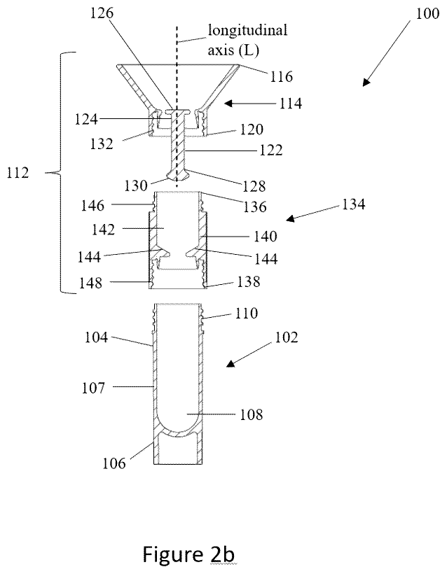

FIGS. 1, 2a-b, 3-b, 4a-b, 7a-d, and 8a-b illustrate a biological sample collection system 100 and components thereof in accordance with one embodiment of the present application. The system 100 includes a containment vessel shown as tube 102, which in the embodiment shown is generally cylindrical in shape. Any suitable containment vessel can be used, such as common sample collection tubes known in the art, or other vials/tubes/containers/vessels having the attributes described herein. The tube 102 comprises a first open end 104 for receiving a biological sample, such as a bodily fluid (e.g. sputum or saliva), a second end 106 comprising a sample storage chamber 108 defined by a wall 107. The tube 102 further comprises a connection member 110 disposed at the first open end 104, which in the illustrated embodiment is external helical threads on an outer surface of wall 107 of the tube 102. As the skilled worker will appreciate, in an alternate embodiment, internal helical threads can be present (not shown).

The tube 102 can be of any desired width, length or thickness as needed, and is made of an inert, durable material, such as polyethylene, polypropylene, polystyrene or related plastic. The tube 102 is ideally self-standing. Optionally, a base can be provided (not shown) for the tube 102 to prevent the system 100 from falling over. The sample storage chamber 108 is suitable for holding a substance such as a liquid, solid, semi-solid, slurry, suspension, powder, colloid, gel, gas, mixtures thereof or the like. The sample storage chamber 108 should have a sufficient void volume to hold the biological sample, plus any desired composition or agent for mixing with the sample.

The wall 107 of the tube 102 should ideally be transparent or translucent to permit viewing of the sample once collected. The wall 107 can be free of any indicia or other markings, and should be suitable to be comfortably handled by the user. However, it may be adorned, if desired, and/or have a grip or a raised texture on an external surface thereof to facilitate handling, or with graduated markings to indicate volume.

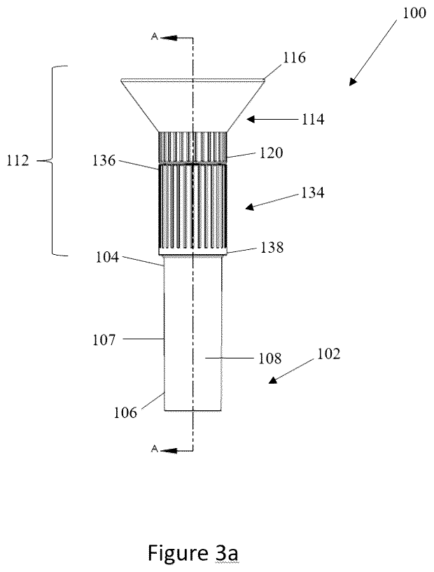

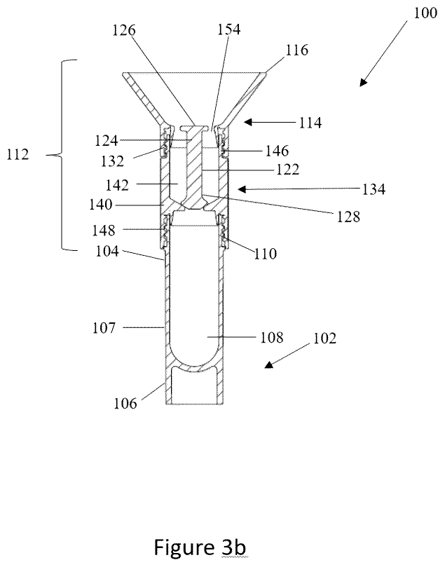

The system 100 further includes a sample collection assembly 112 connected to the first open end 104 of the tube 102. The sample collection assembly 112 comprises a sample collector 114. The sample collector 114 comprises a proximal open end 116 for receiving a biological sample 118, and a distal open end 120 in fluid communication with the proximal open end 116. In one embodiment, the sample collector 114 tapers from the proximal open end 116 to the distal open end 120. In the embodiment shown in FIGS. 1, 2a-b, 3-b, 4a-b and 8a-b, the sample collector 114 is funnel-shaped. The sample collector 114 further comprises a valve stem 122 comprising a first stem end 124 extending from an inner portion 126 of the sample collector 114 (see in particular FIGS. 2b and 3b, and FIGS. 4a-b), the inner portion 126 being disposed proximal to the distal open end 120 of the sample collector 114, and an opposing second stem end 128 comprising a valve face 130, the valve stem 122 extending along a longitudinal axis (L) of the sample collector 114 and in a direction away from the proximal open end 116 of the sample collector 114 (see in particular FIG. 2b). In the embodiment shown, the opposing second stem end 128 extends beyond the distal open end 120 of the sample collector 114. The sample collector 114 further comprises a threaded connection member 132 disposed at the distal open end 120 of the sample collector 114.

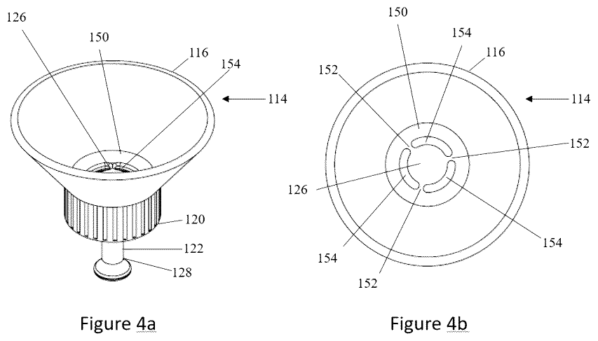

As can best be seen in FIGS. 4a and 4b, the valve stem 122 extends centrally from the distal open end 120 of the sample collector 114, the inner portion 126 of the sample collector 114 being centrally disposed within the sample collector 114, the inner portion 126 of the sample collector 114 being connected to an inner surface 150 of the sample collector by at least one attachment 152. The embodiment shown in FIG. 4b has three such attachments 152. The inner surface 150 of the sample collector 114, the inner portion 126 of the sample collector 114, and the at least one attachment 152 collectively define at least one opening 154 to provide fluid communication between the distal open end 120 of the sample collector 114 and the proximal open end 136 of the sample collection vessel 134. The embodiment shown in FIG. 4b has three such openings 154.

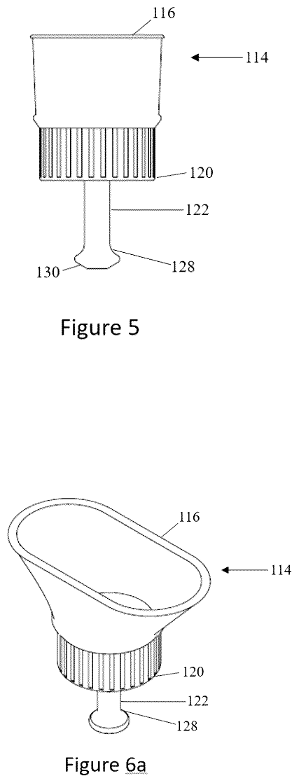

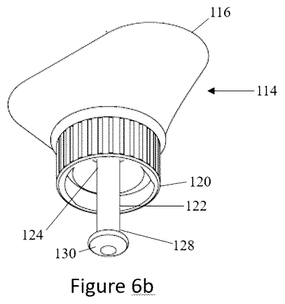

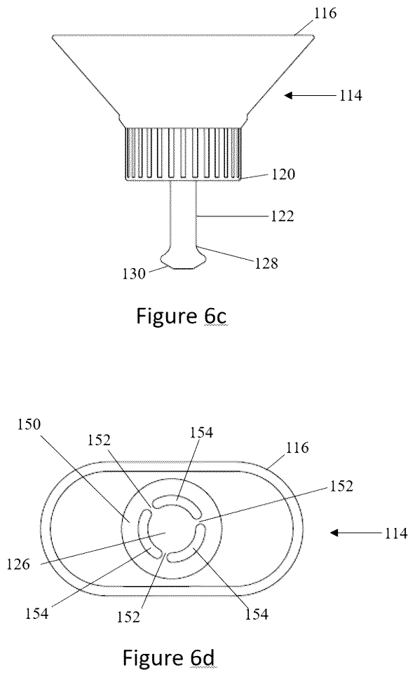

FIGS. 5 and 6a-d illustrate alternate embodiments of the sample collector 114, which differ from the embodiment shown in FIGS. 4a-b in terms of their shape. The embodiment shown in FIGS. 6a-d is similar to that of FIGS. 4a-b, in that the sample collector 114 is funnel-shaped; however, while the proximal open end 116 of the embodiment of FIGS. 4a-b is circular in top view, the proximal open end 116 of the embodiment of FIGS. 6a-d has more of an oblong shape in top view (e.g. stadium- or pill-shaped), which may be advantageous for shipping purposes. FIG. 5 illustrates an alternate embodiment wherein the sample collector 114 has more of a cylindrical shape.

The sample collector 114 can be of any desired width, length or thickness as needed, and can be made of an inert, durable material, such as polyethylene, polypropylene, polystyrene or related plastic, or glass. The collector 114 can be formed from a one-piece mold, or, alternatively, the collector 114 can be formed from separate parts that are assembled to form this portion of the sample collection system 100. Alternatively, in any of the embodiments of the sample collector 114, at least a portion of the sample collector 114 extending from the proximal open end 116 can comprise a flexible material, such as silicone or rubber, to allow this portion of the sample collector 114 to be compressed during shipping, while maintaining the more rigid, durable plastic material in the portion of the sample collector 114 from which the valve stem 122 extends as well as where the threaded connection member 132 is located.

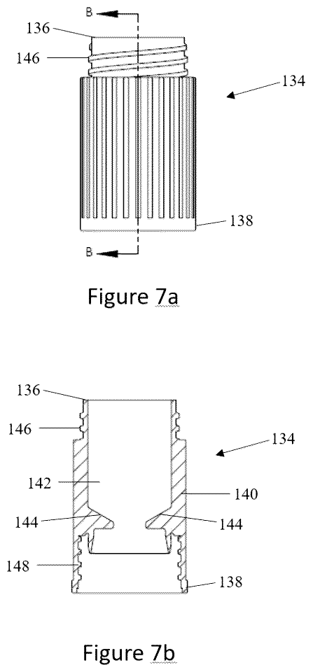

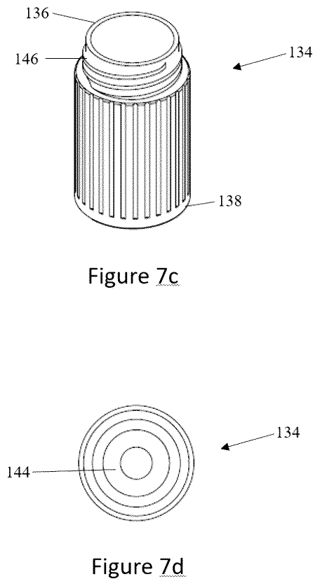

The sample collection assembly 112 further comprises a sample collection vessel 134 in fluid communication with the sample collector 114. The sample collection vessel 134 comprises a proximal open end 136 for receiving the biological sample 118 from the sample collector 114, and a distal open end 138 configured to be in fluid communication with the proximal open end 136. The sample collection vessel 134 has one or more walls 140 defining a sample receiving area 142 and a valve seat 144 (see in particular FIGS. 2b and 7b), wherein the valve seat 144 is complementary to the valve face 130 of the sample collector 114. In one embodiment, the sample receiving area 142 of the sample collection vessel 134 is volumetric and/or contains markings or a fill-to-line, so the donor delivers the correct amount of biological sample for subsequent mixing with the stabilizing composition, preservative, and/or neutralizing agent. As the skilled worker will appreciate, the capacity is scalable to any desired volume. In an embodiment where the biological sample is saliva/sputum, the capacity of the sample receiving area can be in the range of 1-2 mL. In an embodiment where the biological sample is urine, the capacity of the sample receiving area can be in the range of 1-50 mL. In one embodiment the sample receiving area 142 can have a capacity of from about 0.1 mL to about 10 mL of sample.

In one embodiment, the valve face 130 and/or the valve seat 144 can be made from flexible material or an over-mould (e.g. silicone or rubber). For example, if one surface is hard and the other surface is soft, the respective surfaces can “bite” onto one another and form a good seal.

The sample collection vessel 134 also has a threaded connection member 146 disposed at the proximal open end 136, wherein the threaded connection member 146 is complementary to and configured to be engaged with the threaded connection member 132 of the sample collector 114. The sample collection vessel 134 further has a connection member 148 disposed at the distal open end 138, wherein the connection member 148 is complementary to and configured to be engaged with the connection member 110 of the tube 102. In the embodiment shown in the figures, the connection member 110 disposed at the first open end 104 of the tube 102 and the connection member 148 disposed at the distal open end 138 of the sample collection vessel 134 comprise a threaded connection; however, other connection means known to those of skill in the art can also be used, such as a snap-fit or press-fit arrangement.

The sample collection vessel 134 can be of any desired width, length or thickness as needed, and can be made of an inert, durable material, such as polyethylene, polypropylene, polystyrene or related plastic, or glass.

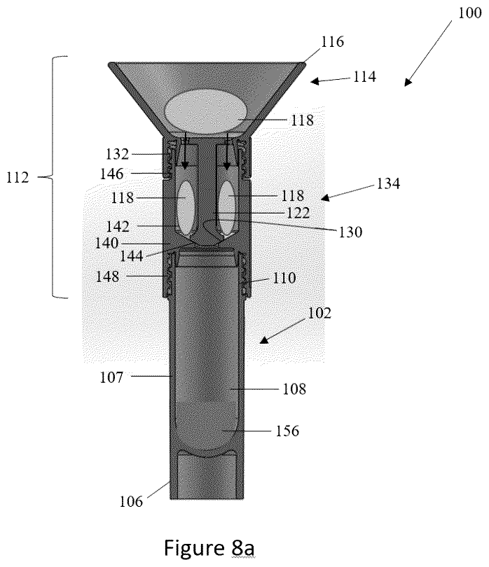

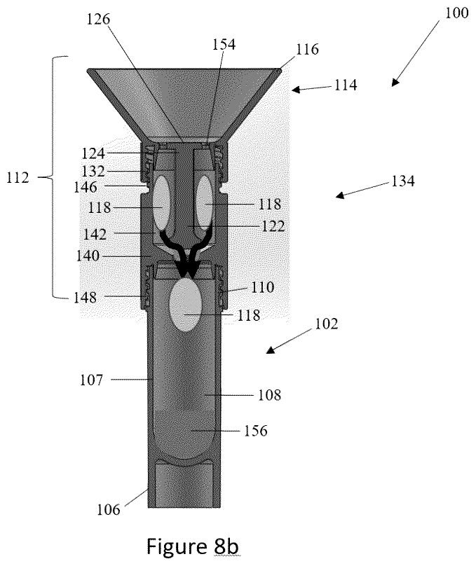

As can be best seen in FIGS. 8a-b, the valve stem 122 comprising the valve face 130, and the valve seat 144 form a valve comprising: (a) a closed configuration, wherein the threaded connection member 132 of the sample collector 114 and the threaded connection member 146 of the sample collection vessel 134 are fully engaged, wherein when the valve is in the closed configuration the valve face 130 forms a sealing engagement with the valve seat 144 (FIG. 8a); and (b) an open configuration, wherein the threaded connection member 132 of the sample collector 114 and the threaded connection member 146 of the sample collection vessel 134 are less than fully engaged, wherein when the valve is in the open configuration the valve face 130 is spaced apart from the valve seat 144 such that the tube 102, the sample collection vessel 134, and the sample collector 114 are in fluid communication with one another (FIG. 8b). In an embodiment, a “click” may be felt by the user and/or a tab may be shown on the sample collector 114 and the sample collection vessel 134 to indicate to the user when the open configuration of the valve has been achieved. In an embodiment, the sealing engagement between the valve face 130 and the valve seat 144 in the closed configuration is fluid-tight.

As shown in FIGS. 8a-b, the sample storage chamber 108 of the tube 102 can comprise a stabilization composition, a preservative, and/or a neutralizing agent 156.

In the embodiment shown in FIGS. 8a-b, the valve is configured to be moved from the closed configuration to the open configuration by rotating the sample collector 114 in a first direction (e.g. counter-clockwise) relative to the sample collection vessel 134, such that the threaded connection members (132, 146) of the sample collector 114 and the sample collection vessel 134 are less than fully engaged. In an embodiment, rotating the sample collector 114 in the first direction relative to the sample collection vessel 134 involves a rotation of about one-quarter turn.

The valve can then be returned to the closed configuration from the open configuration by rotating the sample collector 114 in a second direction (e.g. clockwise) relative to the sample collection vessel 134 to fully engage the threaded connection members (132, 146) of the sample collector 114 and the sample collection vessel 134, wherein the second direction is opposite to the first direction. In an embodiment, rotating the sample collector 114 in the second direction relative to the sample collection vessel 134 comprises a rotation of about one-quarter turn.

Thus, in one embodiment, there is provided a method of preserving a biomolecule and/or neutralizing one or more inhibitors in a biological sample, the method comprising: a) obtaining a biological sample 118; b) obtaining the biological sample collection system 100 as described herein, wherein the valve is in the closed configuration; c) placing the biological sample 118 in the sample collector 114, the sample collector 114 being in fluid communication with the sample collection vessel 134 such that the biological sample 118 is received in the sample receiving area 142 of the sample collection vessel 134 (see FIG. 8a); d) rotating the sample collector 114 in a first direction (e.g. counter-clockwise) relative to the sample collection vessel 134 to move the valve from the closed configuration to the open configuration, such that the tube 102, the sample collection vessel 134, and the sample collector 114 are in fluid communication with one another and the biological sample 118 is received in the sample storage chamber 108 of the tube 102; e) optionally, rotating the sample collector 114 in a second direction (e.g. clockwise) relative to the sample collection vessel 134 to return the valve to the closed configuration, wherein the second direction is opposite to the first direction; and f) mixing the biological sample 118 received in the sample storage chamber 108 of the tube 102 with a stabilizing composition, a preservative, and/or a neutralizing agent 156 in the tube 102 for preserving the biomolecule and/or neutralizing one or more inhibitors within the biological sample 118.

In the case of a biological sample that is saliva/sputum, the biological sample 118 can be placed in the sample collector 114 by expectoration from the mouth of a user/sample donor.

In an embodiment, rotating the sample collector 114 in the first direction relative to the sample collection vessel 134 comprises a rotation of about one-quarter turn. In another embodiment, rotating the sample collector 114 in the second direction relative to the sample collection vessel 134 comprises a rotation of about one-quarter turn.

The biological sample collection system 100 can be transported to the user/sample donor with the valve in the closed configuration, so as to keep the stabilizing composition, preservative, and/or neutralizing agent 156 contained in the tube 102, to avoid contamination of same and to protect the patient/specimen donor from contacting, ingesting and/or spilling the stabilizing composition/preservative/neutralizing agent. Following sample collection, the biological sample collection system 100 can be transported to a laboratory for analysis, again with the valve in the closed configuration, so as to keep the mixture of the stabilizing composition/preservative/neutralizing agent 156 and the biological sample 118 contained in the tube 102 and to avoid contamination of the mixture prior to analysis.

In an alternate embodiment of the sample collection system, the valve stem is a first valve stem and the valve is a first valve, and the sample collection system further comprises a second valve stem comprising: a proximal end extending from and releasably engaged with the opposing second stem end of the first valve stem, and a distal end comprising a second valve face. The second valve stem extends along the longitudinal axis (L) of the sample collector in a direction away from the proximal open end of the sample collector and through an opening defined by the valve seat. The second valve face is configured to engage with the valve seat at an angle relative to an engagement of the valve seat with the valve face of the first valve stem. The second valve stem and the valve seat form a second valve comprising: a second valve open configuration corresponding to the open configuration of the first valve wherein the threaded connection members of the sample collector and the sample collection vessel are less than fully engaged, wherein the second valve face is spaced apart from the valve seat such that the containment vessel, the sample collection vessel, and the sample collector are in fluid communication with one another; a second valve closed configuration wherein the threaded connection members of the sample collector and the sample collection vessel are fully disengaged, resulting in disengagement of the sample collector from the sample collection vessel and disengagement of the proximal end of the second valve stem from the opposing second stem end of the first valve stem, wherein when the second valve is in the second valve closed configuration the second valve face forms a sealing engagement with the valve seat.

In another aspect of the alternate embodiment, the proximal end of the second valve stem is frictionally engaged with the opposing second stem end of the first valve stem within an opening defined by the opposing second stem end of the first valve stem.

In another embodiment, a method of preserving a biomolecule and/or neutralizing one or more inhibitors in a biological sample is provided, the method comprising: a) obtaining a biological sample; b) obtaining the above-noted alternate embodiment of the biological sample collection system, wherein the first valve is in the closed configuration; c) placing the biological sample in the sample collector, the sample collector being in fluid communication with the sample collection vessel such that the biological sample is received in the sample receiving area of the sample collection vessel; d) rotating the sample collector in a first direction (e.g. counter-clockwise) relative to the sample collection vessel to move the first valve from the closed configuration to the open configuration and to move the second valve to the second valve open configuration, such that the containment vessel, the sample collection vessel, and the sample collector are in fluid communication with one another and the biological sample is received in the sample storage chamber of the containment vessel; e) further rotating the sample collector in the first direction relative to the sample collection vessel to move the second valve to the second valve closed configuration, thereby disengaging the sample collector from the sample collection vessel and disengaging the proximal end of the second valve stem from the opposing second stem end of the first valve stem, such that the second valve face forms a sealing engagement with the valve seat; and f) mixing the biological sample received in the sample storage chamber of the containment vessel with a stabilizing composition, a preservative and/or a neutralizing agent in the containment vessel for preserving the biomolecule and/or neutralizing one or more inhibitors within the biological sample. In another embodiment, rotating the sample collector in the first direction relative to the sample collection vessel in step (d) comprises a rotation of about one-quarter turn.

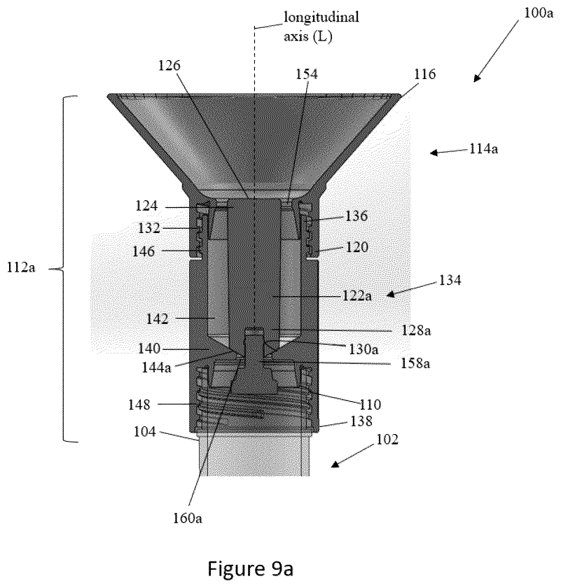

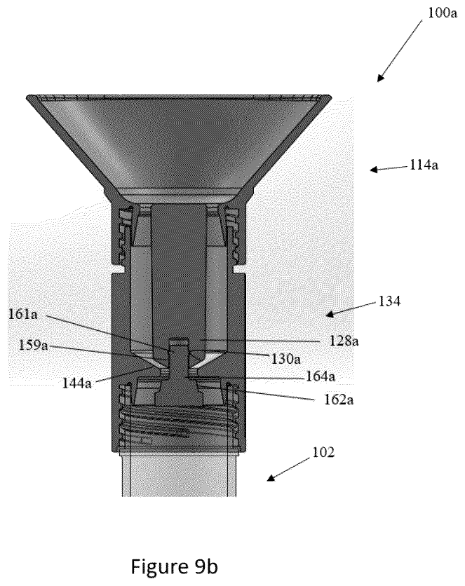

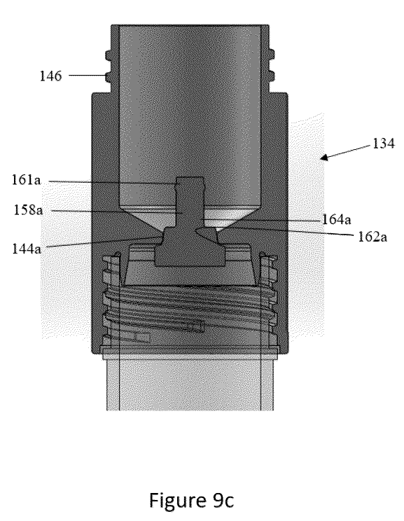

FIGS. 9a-9c illustrate partial views of a biological sample collection system 100a having an alternate embodiment of the sample collector, designated as 114a, wherein elements having the same structure and function as previously described biological sample collection system 100 and sample collector 114 are given the same reference number and will not be described in the same detail again herein, for conciseness. The system 100a includes a sample collection assembly 112a connected to the first open end 104 of the tube 102. The sample collection assembly 112a comprises a sample collector 114a, as noted above. The sample collector 114a comprises a proximal open end 116 for receiving a biological sample (not shown), and a distal open end 120 in fluid communication with the proximal open end 116. The sample collector 114a further comprises a valve stem 122a comprising a first stem end 124 extending from an inner portion 126 of the sample collector 114a, the inner portion 126 being disposed proximal to the distal open end 120 of the sample collector 114a, and an opposing second stem end 128a comprising a valve face 130a, the valve stem 122a extending along a longitudinal axis (L) of the sample collector 114a and in a direction away from the proximal open end 116 of the sample collector 114. In the embodiment shown, the opposing second stem end 128a extends beyond the distal open end 120 of the sample collector 114a. The sample collector 114a further comprises a threaded connection member 132 disposed at the distal open end 120 of the sample collector 114a.

The valve stem 122a extends centrally from the distal open end 120 of the sample collector 114a, the inner portion 126 of the sample collector 114a being centrally disposed within the sample collector 114a, the inner portion 126 of the sample collector 114a being connected to an inner surface of the sample collector by at least one attachment (not shown). As with the embodiment of the sample collector 114, the inner surface of the sample collector 114a, the inner portion 126 of the sample collector 114a, and the at least one attachment collectively define at least one opening 154 to provide fluid communication between the distal open end 120 of the sample collector 114a and the proximal open end 136 of the sample collection vessel 134.

The sample collector 114a can be of any desired width, length or thickness as needed, and can be made of an inert, durable material, such as polyethylene, polypropylene, polystyrene or related plastic, or glass. The collector 114a can be formed from a one-piece mold, or, alternatively, the collector 114a can be formed from separate parts that are assembled to form this portion of the sample collection system 100a. Alternatively, in any of the embodiments of the sample collector 114a, at least a portion of the sample collector 114a extending from the proximal open end 116 can comprise a flexible material, such as silicone or rubber, to allow this portion of the sample collector 114a to be compressed during shipping, while maintaining the more rigid, durable plastic material in the portion of the sample collector 114a from which the valve stem 122a extends as well as where the threaded connection member 132 is located.

The sample collection assembly 112a further comprises a sample collection vessel 134 in fluid communication with the sample collector 114a. The sample collection vessel 134 comprises a proximal open end 136 for receiving the biological sample (not shown) from the sample collector 114a, and a distal open end 138 configured to be in fluid communication with the proximal open end 136. The sample collection vessel 134 has one or more walls 140 defining a sample receiving area 142 and a valve seat 144a, wherein the valve seat 144a is complementary to the valve face 130a of the sample collector 114a.

The sample collection vessel 134 also has a threaded connection member 146 disposed at the proximal open end 136, wherein the threaded connection member 146 is complementary to and configured to be engaged with the threaded connection member 132 of the sample collector 114a. The sample collection vessel 134 further has a connection member 148 disposed at the distal open end 138, wherein the connection member 148 is complementary to and configured to be engaged with the connection member 110 of the tube 102. In the embodiment shown in the figures, the connection member 110 disposed at the first open end 104 of the tube 102 and the connection member 148 disposed at the distal open end 138 of the sample collection vessel 134 comprise a threaded connection; however, other connection means known to those of skill in the art can also be used

The valve stem 122a comprising the valve face 130a, and the valve seat 144a form a valve comprising: (a) a closed configuration (FIG. 9a), wherein the threaded connection member 132 of the sample collector 114a and the threaded connection member 146 of the sample collection vessel 134 are fully engaged, wherein when the valve is in the closed configuration the valve face 130a forms a sealing engagement with the valve seat 144a; and (b) an open configuration (FIG. 9b), wherein the threaded connection member 132 of the sample collector 114a and the threaded connection member 146 of the sample collection vessel 134 are less than fully engaged, wherein when the valve is in the open configuration the valve face 130a is spaced apart from the valve seat 144a such that the tube 102, the sample collection vessel 134, and the sample collector 114a are in fluid communication with one another. In an embodiment, a “click” may be felt by the user and/or a tab may be shown on the sample collector 114a and the sample collection vessel 134 to indicate to the user when the open configuration of the valve has been achieved. In an embodiment, the sealing engagement between the valve face 130a and the valve seat 144a in the closed configuration is fluid-tight.

In the embodiment shown in FIGS. 9a-9c, a second valve stem 158a extends centrally from and in one embodiment is frictionally engaged at its proximal end 161a with the opposing second stem end 128a of the valve stem 122a within an opening 159a defined by the opposing second stem end 128a of the valve stem 122a, where the second valve stem 158a extends through an opening 160a defined by the valve seat 144a. Other means for releasably engaging the second valve stem 158a with the opposing second stem end 128a of the valve stem 122a would be known to the skilled worker. The second valve stem 158a extends along a longitudinal axis (L) of the sample collector 114a and in a direction away from the proximal open end 116 of the sample collector 114. The second valve stem 158a has a second valve face 162a at its distal end 164a that is configured to engage with the valve seat 144a at an angle relative to an engagement of the valve seat 144a with the valve face 130a. Engagement of the second valve face 162a of the second valve stem 158a with the valve seat 144a then closes the valve, and the continued twisting off of the sample collector 114a disengages the second valve stem 158a from the opposing second stem end 128a of the valve stem 122a. The sample collector 114a can then be disengaged from the sample collection vessel 134. The second valve stem 158a will disengage from the opposing second stem end 128a of the valve stem 122a when the downward force exerted by the valve seat 144a on the second valve face 162a exceeds the frictional force maintaining engagement of the second valve stem 158a with the opposing second stem end 128a of the valve stem 122a, thus creating a seal. Upon removal of the sample collector 114a from the sample collection vessel 134, a cap (not shown) could also be used to engage with the threaded connection member 146 of the sample collection vessel 134 to provide an additional means for keeping the admixture of the biological sample with the stabilizing composition, preservative, and/or neutralizing agent (not shown) contained in the tube 102.

In one embodiment, the valve faces 130a/162a and/or the valve seat 144a can be made from flexible material or an over-mould (e.g. silicone or rubber). For example, if one surface is hard and the other surface is soft, the respective surfaces can “bite” onto one another and form a good seal.

In one embodiment, the biological sample collection system 100a is pre-assembled for the user. To assemble the biological sample collection system 100a, the sample collection vessel 134 is first attached to the sample collector 114a (via engagement of connection members 132 and 146) without the second valve stem 158a. The second valve stem 158a is then engaged within the opening 159a defined by the opposing second stem end 128a of the valve stem 122a by insertion through the opening 160a defined by the valve seat 144a. Subsequently, the tube 102 is connected to the sample collection vessel 134 via engagement of connection members 148 and 110.

The biological sample collection system 100/100a described herein is particularly advantageous, as it can be provided to a user/sample donor fully assembled all in one piece, thus avoiding any loose parts. Further, the sample collection system 100/100a operates with only minor manipulations by the user/sample donor (a simple untwisting/twisting action between the sample collector 114/114a and the sample collection vessel 134 following collection of the biological sample), which results in a user-friendly and streamlined method of use for the user/sample donor.

The biological sample can be recovered from the biological sample collection system 100/100a in a laboratory environment by disengaging the sample collection assembly 112/112a from the tube 102.

List of elements and reference numbers

| Ref. No. | Element |
|---|---|
| 100/100a | biological sample collection system |
| 102 | tube (containment vessel) |
| 104 | first open end of tube |
| 106 | second end of tube |
| 107 | wall of tube |
| 108 | sample storage chamber of tube |
| 110 | connection member of tube |
| 112/112a | sample collection assembly |
| 114/114a | sample collector |
| 116 | proximal open end of sample collector |
| 118 | biological sample |
| 120 | distal open end of sample collector |
| 122/122a | valve stem |
| 124 | first stem end |
| 126 | inner portion of the sample collector |
| 128/128a | opposing second stem end |
| 130/130a | valve face |
| 132 | threaded connection member of sample collector |
| 134 | sample collection vessel |
| 136 | proximal open end of the sample collection vessel |
| 138 | distal open end of the sample collection vessel |
| 140 | wall(s) of sample collection vessel |
| 142 | sample receiving area of sample collection vessel |
| 144/144a | valve seat |
| 146 | threaded connection member of sample collection |
| | vessel |
| 148 | connection member disposed at distal open end of |
| | sample collection vessel |
| 150 | inner surface of the sample collector |
| 152 | attachment between inner portion of sample |
| | collector and inner surface of sample collector |
| 154 | opening in sample collector |
| 156 | stabilizing composition/preservative/neutralizing |
| | agent |
| 158a | second valve stem |
| 159a | opening in the opposing second stem end 128a of |
| | the valve stem 122a |
| 160a | opening created by valve seat 144a |
| 161a | proximal end of valve stem 122a |
| 162a | second valve face |
| 164a | distal end of second valve stem |
| L | longitudinal axis of sample collector |

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims. The scope of the claims should not be limited to the preferred embodiments set for the description, but should be given the broadest interpretation consistent with the description as a whole.

1. A biological sample collection system, the system comprising:
a containment vessel comprising:
a first open end for receiving a biological sample,
a second end comprising a sample storage chamber, and
a connection member disposed at the first open end;
a sample collection assembly configured to be connected to the first open end of the containment vessel when the biological sample is being collected, the sample collection assembly comprising:
a sample collector comprising:
a proximal open end for receiving a biological sample;
a distal open end in fluid communication with the proximal open end;
a valve stem comprising:
a first stem end extending from an inner portion of the sample collector, the inner portion being disposed proximal to the distal open end of the sample collector, and
an opposing second stem end comprising a valve face,
the valve stem extending along a longitudinal axis of the sample collector and in a direction away from the proximal open end of the sample collector; and
a threaded connection member disposed at the distal open end of the sample collector; and
a sample collection vessel configured to be in fluid communication with the sample collector when the biological sample is being collected, the sample collection vessel comprising:
a proximal open end for receiving the biological sample from the sample collector;
a distal open end configured to be in fluid communication with the proximal open end;
one or more walls defining a sample receiving area and a valve seat, wherein the valve seat is complementary to the valve face of the sample collector;
a threaded connection member disposed at the proximal open end, wherein the threaded connection member is complementary to and configured to be engaged with the threaded connection member of the sample collector; and
a connection member disposed at the distal open end, wherein the connection member is complementary to and configured to be engaged with the connection member of the containment vessel;
wherein the valve stem and the valve seat form a valve comprising:
a closed configuration wherein the threaded connection members of the sample collector and the sample collection vessel are fully engaged, wherein when the valve is in the closed configuration the valve face forms a sealing engagement with the valve seat; and
an open configuration wherein the threaded connection members of the sample collector and the sample collection vessel are less than fully engaged, wherein when the valve is in the open configuration the valve face is spaced apart from the valve seat such that the containment vessel, the sample collection vessel, and the sample collector are in fluid communication with one another.

2. The sample collection system of claim 1, wherein the valve stem extends centrally from the distal open end of the sample collector, the inner portion of the sample collector being centrally disposed within the sample collector, the inner portion of the sample collector being connected to an inner surface of the sample collector by at least one attachment;
the inner surface of the sample collector, the inner portion of the sample collector, and the at least one attachment collectively defining at least one opening to provide fluid communication between the distal open end of the sample collector and the proximal open end of the sample collection vessel when the biological sample is being collected.

3. The sample collection system of claim 1 or 2, wherein the sealing engagement between the valve face and the valve seat is fluid-tight.

4. The sample collection system of any one of claims 1 to 3, wherein the sample collector is funnel-shaped.

5. The sample collection system of any one of claims 1 to 4, wherein at least a portion of the sample collector extending from the proximal open end comprises a flexible material.

6. The sample collection system of claim 5, wherein the flexible material comprises silicone or rubber.

7. The sample collection system of any one of claims 1 to 6, wherein the connection member disposed at the first open end of the containment vessel and the connection member disposed at the distal open end of the sample collection vessel comprise a threaded connection.

8. The sample collection system of any one of claims 1 to 7, wherein the opposing second stem end extends beyond the distal open end of the sample collector.

9. The sample collection system of any one of claims 1 to 8, wherein the sample receiving area of the sample collection vessel has a capacity of from about 0.1 mL to about 10 mL of sample.

10. The sample collection system of any one of claims 1 to 9, wherein the sample storage chamber of the containment vessel comprises a stabilization composition, a preservative, and/or a neutralizing agent.

11. The sample collection system of any one of claims 1 to 10, wherein the valve is configured to be moved from the closed configuration to the open configuration by rotating the sample collector in a first direction (e.g. counter-clockwise) relative to the sample collection vessel, such that the threaded connection members of the sample collector and the sample collection vessel are less than fully engaged.

12. The sample collection system of claim 11, wherein rotating the sample collector in the first direction relative to the sample collection vessel comprises a rotation of about one-quarter turn.

13. The sample collection system of claim 11 or 12, wherein the valve can be returned to the closed configuration from the open configuration by rotating the sample collector in a second direction (e.g. clockwise) relative to the sample collection vessel to fully engage the threaded connection members of the sample collector and the sample collection vessel,
wherein the second direction is opposite to the first direction.

14. The sample collection system of claim 13, wherein rotating the sample collector in the second direction relative to the sample collection vessel comprises a rotation of about one-quarter turn.

15. The sample collection system of any one of claims 1-14, wherein the valve stem is a first valve stem and the valve is a first valve, the sample collection system further comprising:
a second valve stem comprising:
a proximal end extending from and releasably engaged with the opposing second stem end of the first valve stem, and
a distal end comprising a second valve face;
the second valve stem extending along the longitudinal axis (L) of the sample collector in a direction away from the proximal open end of the sample collector and through an opening defined by the valve seat,
wherein the second valve face is configured to engage with the valve seat at an angle relative to an engagement of the valve seat with the valve face of the first valve stem,
wherein the second valve stem and the valve seat form a second valve comprising:
a second valve open configuration corresponding to the open configuration of the first valve wherein the threaded connection members of the sample collector and the sample collection vessel are less than fully engaged, wherein the second valve face is spaced apart from the valve seat such that the containment vessel, the sample collection vessel, and the sample collector are in fluid communication with one another;
a second valve closed configuration wherein the threaded connection members of the sample collector and the sample collection vessel are fully disengaged, resulting in disengagement of the sample collector from the sample collection vessel and disengagement of the proximal end of the second valve stem from the opposing second stem end of the first valve stem, wherein when the second valve is in the second valve closed configuration the second valve face forms a sealing engagement with the valve seat.

16. The sample collection system of claim 15, wherein the proximal end of the second valve stem is frictionally engaged with the opposing second stem end of the first valve stem within an opening defined by the opposing second stem end of the first valve stem.

17. A method of preserving a biomolecule and/or neutralizing one or more inhibitors in a biological sample, the method comprising:
a) obtaining a biological sample;
b) obtaining the biological sample collection system of any one of claims 1-10, wherein the valve is in the closed configuration;
c) placing the biological sample in the sample collector, the sample collector being in fluid communication with the sample collection vessel such that the biological sample is received in the sample receiving area of the sample collection vessel;
d) rotating the sample collector in a first direction (e.g. counter-clockwise) relative to the sample collection vessel to move the valve from the closed configuration to the open configuration, such that the containment vessel, the sample collection vessel, and the sample collector are in fluid communication with one another and the biological sample is received in the sample storage chamber of the containment vessel;
e) optionally, rotating the sample collector in a second direction (e.g. clockwise) relative to the sample collection vessel to return the valve to the closed configuration, wherein the second direction is opposite to the first direction; and
f) mixing the biological sample received in the sample storage chamber of the containment vessel with a stabilizing composition, a preservative and/or a neutralizing agent in the containment vessel for preserving the biomolecule and/or neutralizing one or more inhibitors within the biological sample.

18. The method of claim 17, wherein rotating the sample collector in the first direction relative to the sample collection vessel comprises a rotation of about one-quarter turn.

19. The method of claim 17 or 18, wherein rotating the sample collector in the second direction relative to the sample collection vessel comprises a rotation of about one-quarter turn.

20. A method of preserving a biomolecule and/or neutralizing one or more inhibitors in a biological sample, the method comprising:
a) obtaining a biological sample;
b) obtaining the biological sample collection system of claim 15 or 16, wherein the first valve is in the closed configuration;
c) placing the biological sample in the sample collector, the sample collector being in fluid communication with the sample collection vessel such that the biological sample is received in the sample receiving area of the sample collection vessel;
d) rotating the sample collector in a first direction (e.g. counter-clockwise) relative to the sample collection vessel to move the first valve from the closed configuration to the open configuration and to move the second valve to the second valve open configuration, such that the containment vessel, the sample collection vessel, and the sample collector are in fluid communication with one another and the biological sample is received in the sample storage chamber of the containment vessel;
e) further rotating the sample collector in the first direction relative to the sample collection vessel to move the second valve to the second valve closed configuration, thereby disengaging the sample collector from the sample collection vessel and disengaging the proximal end of the second valve stem from the opposing second stem end of the first valve stem, such that the second valve face forms a sealing engagement with the valve seat; and
f) mixing the biological sample received in the sample storage chamber of the containment vessel with a stabilizing composition, a preservative and/or a neutralizing agent in the containment vessel for preserving the biomolecule and/or neutralizing one or more inhibitors within the biological sample.

21. The method of claim 20, wherein rotating the sample collector in the first direction relative to the sample collection vessel in step (d) comprises a rotation of about one-quarter turn.
